(19) United States
(12) Patent Application Publication
(10) Pub. No.: US 2006/0258020 A1
(43) Pub. Date: Nov. 16, 2006

(54) Sanitary utensil with functions of immuno-chromatographic test and indication

(76) Inventors: Yi-Feng Kao, Sanchong City (TW); Ching-Yu Lin, Yilan City (TW); Peter Chan, Taipei (TW); Li-Te Chin, Taipei (TW); Ying-Chih Wang, Yilan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/125,227

(22) Filed: May 10, 2005

(51) Int. Cl. G01N 33/558

(52) U.S. Cl. 436/514

(57) ABSTRACT

The present invention discloses a sanitary utensil with functions of immuno-chromatographic test and indication, which comprises at least one immuno-chromatographic test piece and at least one cover having an open end. The immuno-chromatographic test piece further comprises a test starting portion, an indicating portion and a test stopping portion. Furthermore, the cover is used to protect the test stopping portion, the indicating portion and part of the test starting portion, so that the bio-specimen is allowed to enter the immuno-chromatographic testing piece only from the test starting portion.

### BACKGROUND OF THE INVENTION

1. Field of the Invention

The present invention is generally related to a sanitary utensil, and more particularly to a sanitary utensil with functions of immuno-chromatographic test and indication.

2. Description of the Prior Art

Immunoassays, which depend on the specific interaction between antigen and antibody, have been developed and used to detect trace amounts of antigen and antibody in bio-specimens by prepared antigen and antibody. If the prepared antigen and antibody are labeled by dectable markers (such as: radionuclide, fluorescent material, enzymes, latex, colloid gold and carbon black), it is provided a better way by detecting the markers on the immune complex (antigen bound to antibody), rather than detecting the immune complex itself. Through this biological amplification of the mentioned markers, the sensitivity of immunoassay is dramatically increased.

immunochromatographic test (ICT) is a rapid, one step, often may easily be handled by less-trained personnel, immunoassay method. It provides visual evidence of the presence of an analyte in a liquid sample. It is more suitable to be applied in point-of-care testing (POCT) than the other immunoassays which are generally multi-steps and complex. The principle of ICT is base on immunoaffinity partition of the analyte among other molecules in sample which are migrating laterally through a porous membrane by capillary phenomenon. Detection of the appearance and disappearance of chromogenic analyte can use either naked eyes (qualitative), or a photometer to distinguish the sample concentration (quantitative) at the end of the test.

The first commercial product of immunochromatographic test (ICT) is Clearview™ in 1988, which is “sandwich” type, labeled with blue latex particles. Clearview™ is used in pregnancy test for detecting human chorionic gonadotropin (hCG) in urine. Afterwards, many companies provide varies immunochromatographic test devices in the market. A general immunochromatographic test piece is shown in FIG. 1, and the test piece comprises a sample pad 110, conjugated pad 120, membrane 130, indication structure 140 and absorbent pad 150. The indication structure 140 can be one test line (not shown in FIG. 1) or a combination of test line 140A and control line 140B.

Conventional immunochromatographic test devices are applied for specimens of stool, urine, blood and sputum. For example, common blood test is an invasive sampling method, which is to make a small prick in the tip of the finger by a blood collecting needle and then squeeze the blood out. Besides, some conventional immunochromatographic test devices, such as blood and stool, are usually provided with other sampling means, which resulting much inconvenience in practice. Therefore, there is still a need to develope new immunochromatographic test devices corresponding to broaden the detecting range of infectious agents and to provide function of sampling.

### SUMMARY OF THE INVENTION

In accordance with the present invention, a new sanitary utensil with functions of immuno-chromatographic test and indication is provided that substantially overcomes the drawbacks of the above problems mentioned from the conventional system. The present invention is appropriate for various infectious agents (antibody or protein) in female's secretion. Furthermore, this invention can not only keep the clean status of body but also detect the bio-specimens of body in same time.

Generally speaking, conventional immunochromatographic test takes a period of time to finish the following two steps: The first step is antigen binding to antibody to form immune complex, and the second step is immune complex migrating to indication structure by capillary phenomenon. For example, the blood test takes 20 to 30 minutes. On the contrast, this invention can not only keep the clean status of body but also detect the bio-specimens of body in same time without providing other sampling means. Additionally, this invention also provides functions of disease detecting and disease monitoring. Moreover, the provided sanitary utensil with functions of immuno-chromatographic test and indication gives facilities for home-based sample collection and renders users conveniently to mail or transport used sanitary utensil with functions of immuno-chromatographic test and indication to medical laboratories for further bio-analysis. Therefore, this present invention does have the economic advantages for industrial applications.

Accordingly, the present invention discloses a sanitary utensil with functions of immuno-chromatographic test and indication, which comprises at least one immuno-chromatographic test piece and at least one cover having an open end. The immuno-chromatographic test piece further comprises a test starting portion, an indicating portion and a test stopping portion. Furthermore, the cover is used to protect the test stopping portion, the indicating portion and part of the test starting portion, so that the bio-specimen is allowed to enter the immuno-chromatographic testing piece only from the test starting portion.

### BRIEF DESCRIPTION OF THE DRAWINGS

FIG. 1 is a schematic diagram of an immunochromatographic test piece according to prior art;

FIG. 2A is a schematic diagram of a sanitary utensil with functions of immuno-chromatographic test and indication, in accordance with a preferred example of the embodiment;

FIG. 2B is a schematic diagram of a sanitary utensil with functions of immuno-chromatographic test and indication, in accordance with a first combination of another preferred example of the embodiment;

FIG. 2C is a schematic diagram of a sanitary utensil with functions of immuno-chromatographic test and indication, in accordance with a second combination of another preferred example of the embodiment;

FIG. 2D is a schematic diagram of a sanitary utensil with functions of immuno-chromatographic test and indication, in accordance with a second combination of another preferred example of the embodiment;

FIG. 2E is a schematic diagram of a sanitary utensil with functions of immuno-chromatographic test and indication, in accordance with a second combination of another preferred example of the embodiment;

FIG. 2F is a schematic diagram of a sanitary utensil with functions of immuno-chromatographic test and indication, in accordance with a third combination of another preferred example of the embodiment;

FIG. 2G is a schematic diagram of a sanitary utensil with functions of immuno-chromatographic test and indication, in accordance with a third combination of another preferred example of the embodiment;

FIG. 2H is a three dimensional view of FIG. 2G;

FIG. 21 is a schematic diagram of a sanitary utensil with functions of immuno-chromatographic test and indication, in accordance with a first combination of still another preferred example of the embodiment;

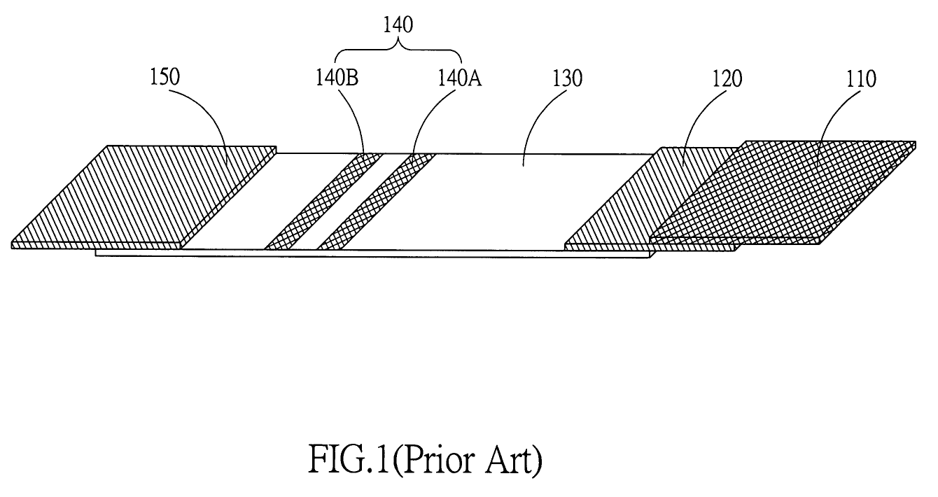

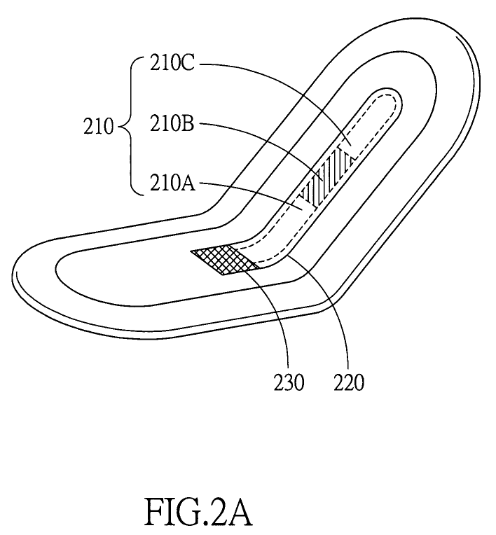

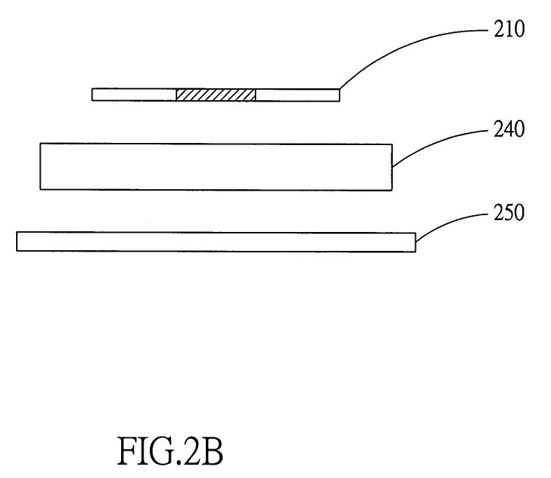

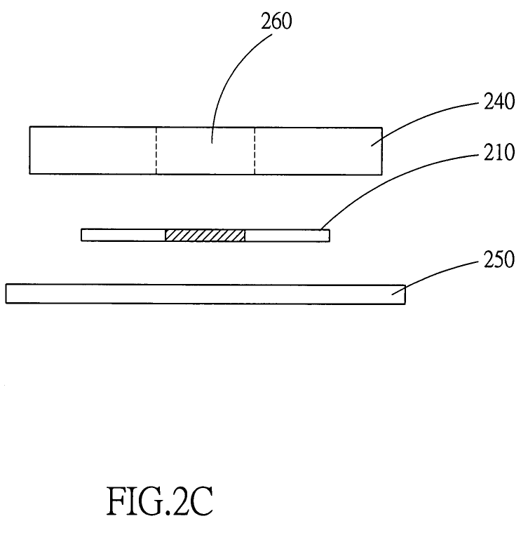

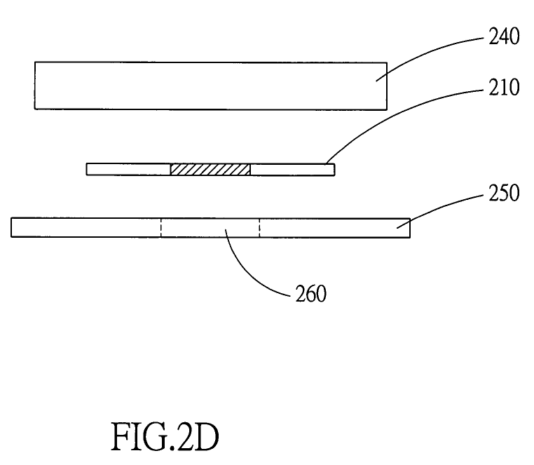

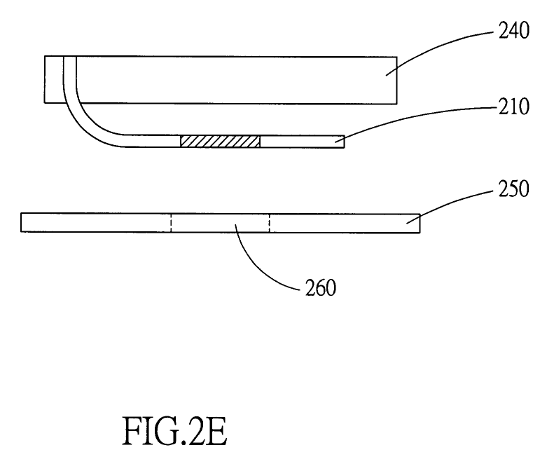

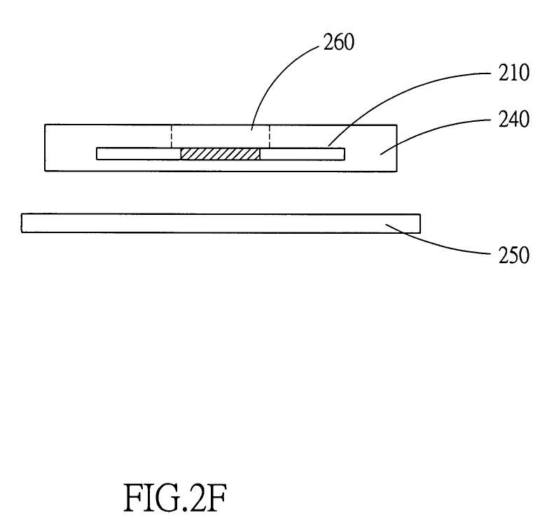

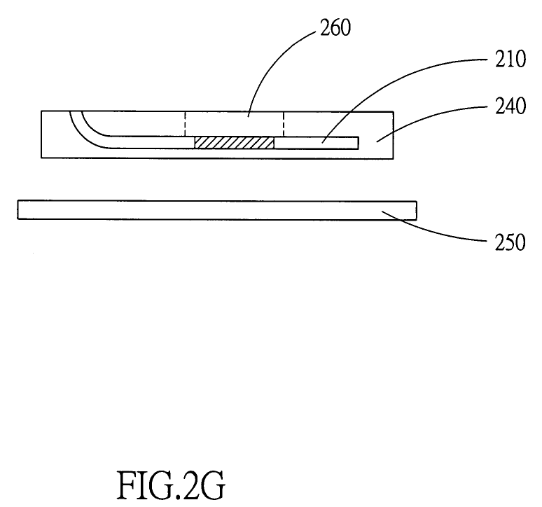

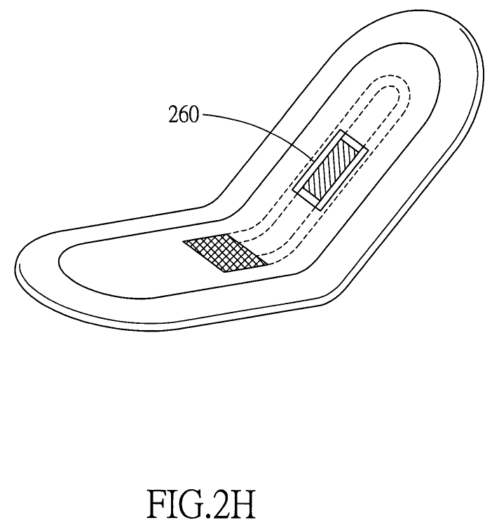

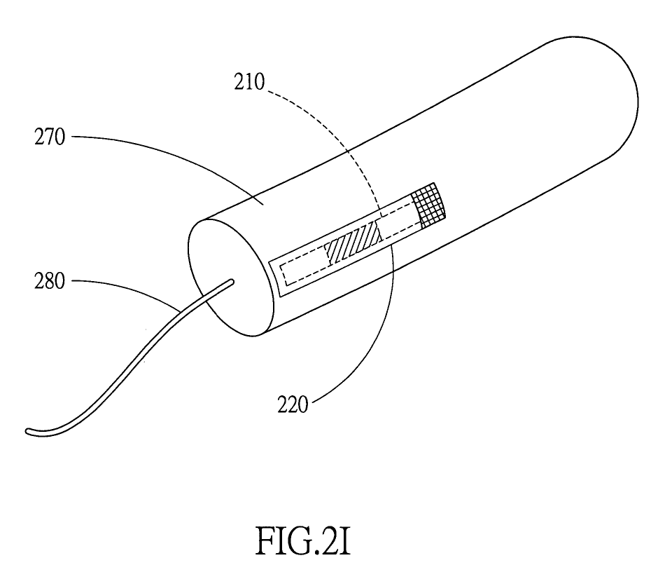

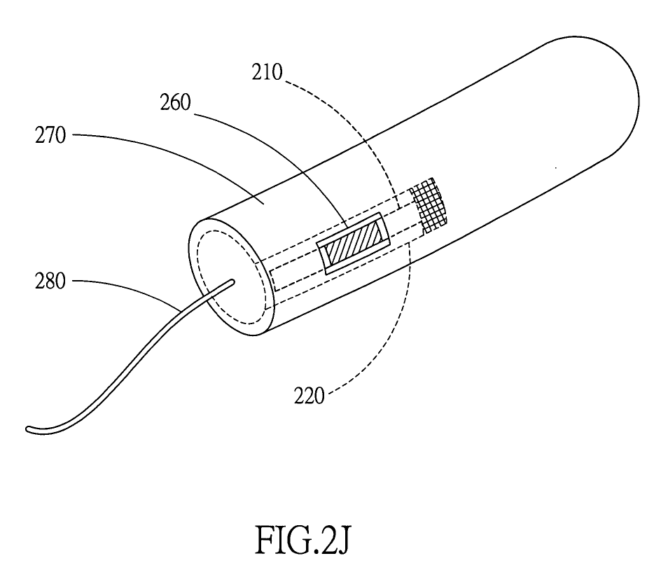

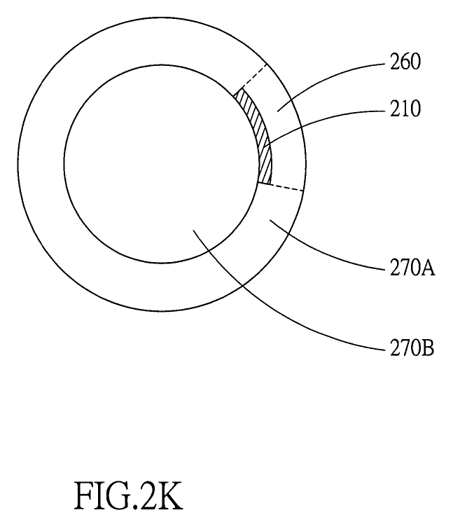

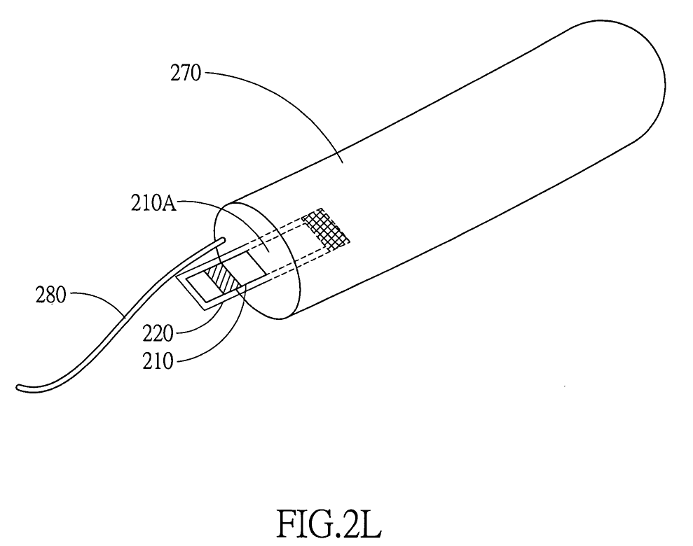

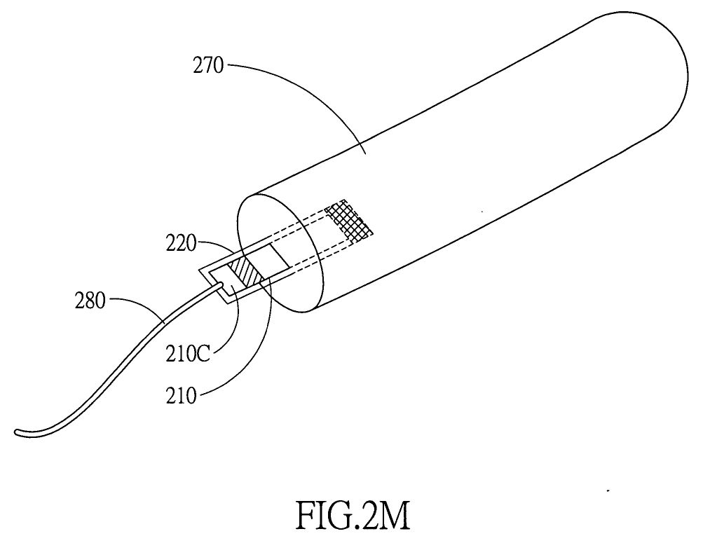

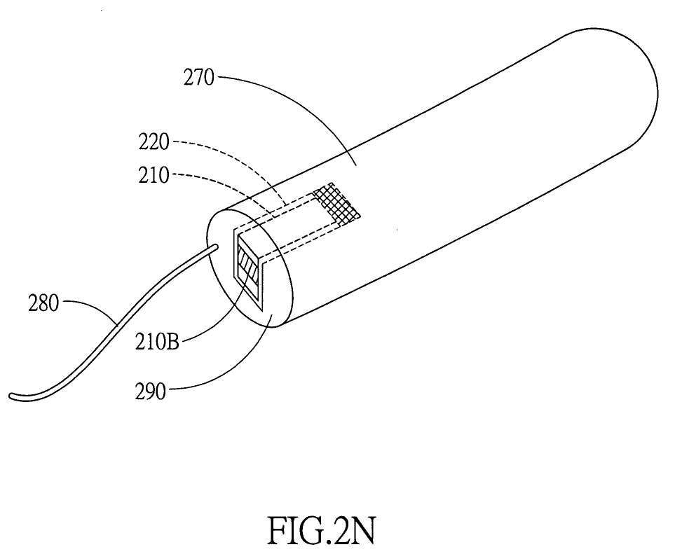

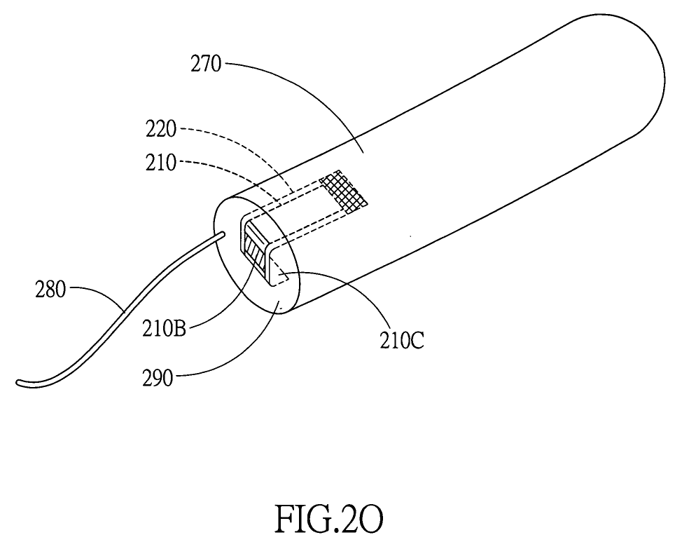

FIG. 2J is a schematic diagram of a sanitary utensil with functions of immuno-chromatographic test and indication, in accordance with a second combination of still another preferred example of the embodiment;

FIG. 2I is a cross-sectional view of FIG. 2K;

FIG. 2L is a schematic diagram of a sanitary utensil with functions of immuno-chromatographic test and indication, in accordance with a third combination of still another preferred example of the embodiment;

FIG. 2M is a schematic diagram of a sanitary utensil with functions of immuno-chromatographic test and indication, in accordance with a third combination of still another preferred example of the embodiment;

FIG. 2N is a schematic diagram of a sanitary utensil with functions of immuno-chromatographic test and indication, in accordance with a fourth combination of still another preferred example of the embodiment; and

FIG. 2O is a schematic diagram of a sanitary utensil with functions of immuno-chromatographic test and indication, in accordance with a fifth combination of still another preferred example of the embodiment.

### DESCRIPTION OF THE PREFERRED EMBODIMENTS

What is probed into the invention is a sanitary utensil with functions of immuno-chromatographic test and indication. Detailed descriptions of the production, structure and elements will be provided in the following in order to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a preferred embodiment of the present invention, a sanitary utensil with functions of immuno-chromatographic test and indication is disclosed, wherein the sanitary utensil comprises at least one immuno-chromatographic test piece and at least one cover having an open end. The immuno-chromatographic test piece further comprises a test starting portion, an indicating portion and a test stopping portion. Furthermore, the cover is used to protect the test stopping portion, the indicating portion and part of the test starting portion, so that the bio-specimen is allowed to enter the immuno-chromatographic testing piece only from the test starting portion.

In this embodiment, the mentioned bio-specimen is selected as any one or any combination of the following: blood, menstrual blood, cervicovaginal secretion, urethral secretion, venereal secretion, cells and tissues shed from reproductive tract and gastral secretion. The above-mentioned sanitary utensil is one of the following: swab, gauze, panty shield, pad, tampon, pantyliner, diaper, toilet paper, or other absorbent. Furthermore, the red blood cells, with size from 6 μm to 8 μm, give the blood its red color, for red blood cells are filled with a special red colored molecule called hemoglobin. Therefore, if the bio-specimen is blood, the test starting portion further comprises a filter to keep the red blood cells or tissues of the bio-specimen from entering the immuno-chromatographic test piece, so as to avoid incorrect result indication. The preferred pore size of the mentioned filter is less than 6 μm. Moreover, if the bio-specimen is too viscous, it might take a long time for chromatography, or even fail to perform chromatography. Therefore, the sanitary utensil disclosed in this invention further comprises a container for storing diluents. The operating procedure is as following: first, the viscous bio-specimen contacts with the test starting portion of the immuno-chromatographic test piece. After decades of seconds, the diluent is discharged from the container to the test starting portion to provide a higher liquid pressure, whereupon the immune complex migrates to the indicating portion more quickly, and the time of chromatography is reduced.

Referring to FIG. 2A, in a preferred example of this embodiment, the sanitary utensil comprises at least one immuno-chromatographic test piece 210, at least one cover having an open end 220 and a filter 230. The immuno-chromatographic test piece 210 further comprises a test starting portion 210A, an indicating portion 210B and a test stopping portion 210C. Additionally, the cover 220 is used to protect the test stopping portion 210C, the indicating portion 210B and part of the test starting portion 210A, so that the bio-specimen is allowed to enter the immuno-chromatographic testing piece 210 only from the test starting portion 210A. The material of the mentioned cover 220 is transparent and/or non-permeating. Moreover, the filter 230 covers the last uncovered portion of the test starting portion 210A by the cover 220, and the filter 230 connects with the cover 220 tightly. The mentioned filter 230 is used to keep the red blood cells or tissues of the bio-specimen from entering the immuno-chromatographic test piece, and the preferred pore size of the filter 230 is less than 6 μm.

In another preferred example of this embodiment, the sanitary utensil further comprises a absorbing structure 240 and a leakage-proof structure 250, wherein there are three combinations among the immuno-chromatographic test piece 210, the cover having an open end 220, the absorbing structure 240 and the leakage-proof structure 250 (for clear presentation of elements, the cover 220 is not shown in FIG. 2B to FIG. 2G). Referring to FIG. 2A and FIG. 2B, the first combination is that the leakage-proof structure 250 is located at one side of the absorbing structure 240, and the immuno-chromatographic test piece 210 is located at the other side of the absorbing structure 240. Referring to FIG. 2C to FIG. 2E, the second combination is that the immuno-chromatographic test piece 210 is located between the absorbing structure 240 and the leakage-proof structure 250, wherein the absorbing structure 240 can comprise a view window 260 (as shown in FIG. 2C), or the leakage-proof structure 250 can comprise a view window 260 (as shown in FIG. 2D), whereupon the user can obtain the test result from the indicating portion 210B of the immuno-chromatographic test piece. Besides, referring to FIG. 2E, a preferred design is that the immuno-chromatographic test piece 210 contacts one side of the absorbing structure 240, and the end of the test starting portion 210A extends to the other side of the absorbing structure 240, so that the bio-specimen is allowed to enter the immuno-chromatographic testing piece 210 only from the test starting portion 210A. Referring to FIG. 2F, the third combination is that the leakage-proof structure 250 is located at one side of the absorbing structure 240, and the immuno-chromatographic test piece 210 is embedded in the absorbing structure 240. Besides, referring to FIG. 2G and FIG. 2H, a preferred design is that the end of the test starting portion 210A extends to the other side of the absorbing structure 240, so that the bio-specimen is allowed to enter the immuno-chromatographic testing piece 210 only from the test starting portion 210A. Additionally, the absorbing structure 240 further comprises a view window 260 on the other side of the absorbing structure 240, whereupon the user can obtain the test result from the indicating portion 210B of the immuno-chromatographic test piece 210.

In still another preferred example of this embodiment, the sanitary utensil further comprises a elongated structure 270 and a pulling element 280, wherein there are five combinations among the immuno-chromatographic test piece 210, the cover having an open end 220, the elongated structure 270 and the pulling element 280. Referring to FIG. 2I, the first combination is that the immuno-chromatographic test piece 210 is located outside the elongated structure 270. Referring to FIG. 2J and FIG. 2K, the second combination is that the elongated structure further 270 further comprises a first absorbing layer 270A and a second absorbing layer 270B, wherein the first absorbing layer 270A is located outside the second absorbing layer 270B, and the immuno-chromatographic test piece 210 is located between the first absorbing layer 270A and the second absorbing layer 270B. Besides, a preferred design is that the first absorbing layer 270A comprises a view window 260, whereupon the user can obtain the test result from the indicating portion 210B of the immuno-chromatographic test piece 210. Referring to FIG. 2L, the third combination is that the immuno-chromatographic test piece 210 contacts the elongated structure 270 by the test starting portion 210A. The word “contact” means the end of the test starting portion 210A connects to the elongated structure 270, or the end of the test starting portion 210A is embedded in the elongated structure 270. Additionally, referring to FIG. 2M, the pulling element 280 can be embedded in the end of the test stopping portion 210C. Referring to FIG. 2N, the fourth combination is that the sanitary utensil comprises an elongated structure 270 with a bottom surface and a pulling element 280 thereon, wherein the immuno-chromatographic test piece 210 is L-shaped, and the indicating portion 210B overlies the bottom surface of the elongated structure 270. Finally, referring to FIG. 20, the fifth combination is that the sanitary utensil comprises an elongated structure 270 with a bottom surface and a pulling element 280 thereon, wherein the immuno-chromatographic test piece 210 is U-shaped; part of test stopping portion 210C is embedded in the elongated structure 270, and the indicating portion 210B is substantially parallel to the bottom surface of the elongated structure 270.

In the above preferred embodiments, the present invention can not only keep the clean status of body but also detect the bio-specimens of body in same time without providing other sampling means. Additionally, this invention also provides functions of disease detecting and disease monitoring. Moreover, the provided sanitary utensil with functions of immuno-chromatographic test and indication gives facilities for home-based sample collection and renders users conveniently to mail or transport used sanitary utensil with functions of immuno-chromatographic test and indication to medical laboratories for further bio-analysis. Therefore, this present invention does have the economic advantages for industrial applications.

To sum up, the present invention discloses a sanitary utensil with functions of immuno-chromatographic test and indication, which comprises at least one immuno-chromatographic test piece and at least one cover having an open end. The immuno-chromatographic test piece further comprises a test starting portion, an indicating portion and a test stopping portion. Furthermore, the cover is used to protect the test stopping portion, the indicating portion and part of the test starting portion, so that the bio-specimen is allowed to enter the immuno-chromatographic testing piece only from the test starting portion.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A sanitary utensil with functions of immuno-chromatographic test and indication, comprising:
at least one immuno-chromatographic test piece, wherein said immuno-chromatographic test piece comprises a test starting portion, an indicating portion and a test stopping portion; and
at least one cover having an open end, wherein said cover is used to protect said test stopping portion, said indicating portion and part of said test starting portion, so that the bio-specimen is allowed to enter said immuno-chromatographic testing piece only from said test starting portion.

2. The sanitary utensil according to claim 1, wherein said test starting portion further comprises a filter to keep the red blood cells or tissues of the bio-specimen from entering said immuno-chromatographic test piece.

3. The sanitary utensil according to claim 1, wherein said test starting portion further comprises a filter with pore size less than 6 μm.

4. The sanitary utensil according to claim 1, wherein the material of said cover is transparent.

5. The sanitary utensil according to claim 1, wherein the material of said cover is non-permeating.

6. The sanitary utensil according to claim 1, further comprising an absorbing structure and a leakage-proof structure, wherein said leakage-proof structure is located at one side of said absorbing structure, and said immuno-chromatographic test piece is located at the other side of said absorbing structure.

7. The sanitary utensil according to claim 1, further comprising an absorbing structure and a leakage-proof structure, wherein said immuno-chromatographic test piece is located between said absorbing structure and said leakage-proof structure.

8. The sanitary utensil according to claim 7, wherein said absorbing structure further comprises a view window, whereupon the user can obtain the test result from said indicating portion of said immuno-chromatographic test piece.

9. The sanitary utensil according to claim 7, wherein said leakage-proof structure further comprises a view window, whereupon the user can obtain the test result from said indicating portion of said immuno-chromatographic test piece.

10. The sanitary utensil according to claim 7, wherein said immuno-chromatographic test piece contacts one side of said absorbing structure, and the end of said test starting portion extends to the other side of said absorbing structure, so that the bio-specimen is allowed to enter said immuno-chromatographic testing piece only from said test starting portion.

11. The sanitary utensil according to claim 1, further comprising an absorbing structure and a leakage-proof structure, wherein said leakage-proof structure is located at one side of said absorbing structure, and said immuno-chromatographic test piece is embedded in said absorbing structure.

12. The sanitary utensil according to claim 11, wherein the end of said test starting portion extends to the other side of said absorbing structure, so that the bio-specimen is allowed to enter said immuno-chromatographic testing piece only from said test starting portion.

13. The sanitary utensil according to claim 11, wherein said absorbing structure further comprises a view window on the other side of said absorbing structure, whereupon the user can obtain the test result from said indicating portion of said immuno-chromatographic test piece.

14. The sanitary utensil according to claim 1, further comprising an elongated structure, and said immuno-chromatographic test piece is located outside said elongated structure.

15. The sanitary utensil according to claim 1, further comprising a elongated structure with a first absorbing layer and a second absorbing layer, wherein said first absorbing layer is located outside said second absorbing layer, and said immuno-chromatographic test piece is located between said first absorbing layer and said second absorbing layer.

16. The sanitary utensil according to claim 1, wherein said first absorbing layer comprises a view window, whereupon the user can obtain the test result from said indicating portion of said immuno-chromatographic test piece.

17. The sanitary utensil according to claim 1, further comprising an elongated structure with a bottom surface and a pulling element thereon, wherein said immuno-chromatographic test piece contacts said elongated structure by said test starting portion.

18. The sanitary utensil according to claim 17, wherein said immuno-chromatographic test piece is L-shaped, and said indicating portion overlies said bottom surface of said elongated structure.

19. The sanitary utensil according to claim 17, wherein said immuno-chromatographic test piece is U-shaped; part of test stopping portion is embedded in said elongated structure, and said indicating portion is substantially parallel to said bottom surface of said elongated structure.

20. The sanitary utensil according to claim 1, further comprising a container for storing diluents.
